(19) United States
(12) Patent Application Publication
(10) Pub. No.: US 2010/0125281 A1
(43) Pub. Date: May 20, 2010

(54) CARDIAC PACING LEAD AND DELIVERY SHEATH

(75) Inventors: Jason Jacobson, Chicago, IL (US); Jason Rubenstein, Naperville, IL (US); Michael Kim, Wilmette, IL (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/620,185

(22) Filed: Nov. 17, 2009

Related U.S. Application Data
(60) Provisional application No. 61/115,339, filed on Nov. 17, 2008.

(51) Int. Cl. A61B 17/00; A61N 1/05

(52) U.S. Cl. 606/129; 607/127

(57) ABSTRACT

The present invention provides compositions and methods related to epicardial pacing systems. In particular, the present invention provides a novel delivery sheath and cardiac pacing lead to be deployed in the pericardial space via a percutaneous approach.

The present application claims priority to U.S. Provisional application 61/115,339, filed Nov. 17, 2008, which is herein incorporated by reference in its entirety.

### FIELD OF THE INVENTION

The present invention provides compositions and methods related to epicardial pacing systems. In particular, the present invention provides a novel delivery sheath and epicardial cardiac pacing lead to be deployed in the pericardial space via a percutaneous approach.

### BACKGROUND OF THE INVENTION

Endocardial pacing systems are commonly used in modern medicine for a wide variety of rhythm disorders. These pacing leads are delivered to the cardiac endocardial surface by means of percutaneous venous entry, usually through the subclavian vein and into the coronary sinus. However, often this is impossible for a variety of reasons such as venous stenosis or tortuous anatomy. In these cases, the current alternative is epicardial leads, placed surgically via open thoractomy. This commonly occurs with left ventricular pacing leads, but may also occur with right atrial or right ventricular leads. This is a less favorable approach, given high peri-operative risks and the need for general anesthesia.

### SUMMARY

In some embodiments, the present invention provides a system comprising (a) an epicardial pacing lead, wherein said epicardial pacing lead comprises a bipolar configuration with respect to the main lead axis, one or more screw-in electrodes, a gearing mechanism, and a rotation shaft; and (b) a delivery sheath, wherein said delivery sheath comprises a cylindrical cover, one or more mapping electrodes, and an orientation balloon; wherein the epicardial pacing lead is configured to reside within the delivery sheath, and said delivery sheath is configured to be removed over said epicardial pacing lead. In some embodiments the screw-in electrodes are configured to extend orthogonally to the main lead axis. In some embodiments, the screw-in electrodes are spaced 1-20 mm apart. In some embodiments, the screw-in electrodes are spaced 5-15 mm apart. In some embodiments, the screw-in electrodes are spaced 10 mm apart. In some embodiments, the gearing mechanism is configured to advance the screw-in electrode orthogonally to the main lead axis. In some embodiments, the rotation shaft is configured to turn the gearing mechanism. In some embodiments, the delivery sheath is steerable via controls at the sheath handle. In some embodiments, the delivery sheath is bidirectionally steerable. In some embodiments, the mapping electrodes sit above the lead electrodes and have the same spacing as the lead electrodes. In some embodiments, the orientation balloon comprises flat pancake-shaped balloon at the sheath tip (i.e., the depth is less than the width; e.g., by a ratio of 2:1, 3:1, 4:1, 5:1, etc.). In some embodiments, the balloon is a standard inflatable percutaneous intervention balloon (e.g., a venoplasty balloon). In some embodiments, the balloon is configured to adjust to the shape of a tissue region. In some embodiments, the balloon may be partially or fully inflated or deflated. In some embodiments, a pancake-shaped balloon is wider than it is deep (e.g., 1.5× wider than deep; 2× wider than deep; 5× wider than deep; 10× wider than deep; 25× wider than deep). In some embodiments, the balloon is tall and narrow (e.g., 1.5× taller than wide; 2× taller than wide; 3× taller than wide; 5× taller than wide; 10× taller than wide; 25× taller than wide). In some embodiments, the orientation balloon is configured to maintain the proper orientation of the sheath within the flat pericardial space. In some embodiments, the delivery sheath is configured to be removed over the epicardial pacing lead after being split by a sheath cutter. In some embodiments, the delivery sheath is configured to be slightly larger than epicardial pacing lead, thereby allowing said sheath to be removed backwards over the epicardial pacing lead.

In some embodiments, the present invention provides a method comprising: inserting a delivery sheath containing an epicardial pacing lead into the pericardium, positioning the delivery sheath in the proper position over the cardiac muscle, testing for proper position of the delivery sheath using mapping electrodes on the outer surface of the delivery sheath, removing the delivery sheath from the epicardial pacing lead, and engaging the epicardial pacing lead with the cardiac muscle by screw-in electrodes positioned at the end of the epicardial pacing lead. In some embodiments, inserting comprises steering the delivery sheath through the pericardium via handles on the sheath handle. In some embodiments, a flat inflatable orientation balloon at the sheath tip is used to maintain proper orientation of said delivery sheath within the flat pericardial space. In some embodiments, testing for proper position of delivery sheath using mapping electrodes comprises testing pacing thresholds and sensing in a fast and non-destructive manner. In some embodiments, removing the delivery sheath from the epicardial pacing lead comprises splitting the delivery sheath. In some embodiments, removing said delivery sheath from the epicardial pacing lead comprises withdrawing the delivery sheath backwards over the epicardial pacing lead. In some embodiments, engaging the epicardial pacing lead with the cardiac muscle comprises screwing the two screw-in electrodes into the cardiac muscle. In some embodiments, the screw-in electrodes are turned by a gearing mechanism within the epicardial pacing lead. In some embodiments, the gearing mechanism is turned by a rotation shaft within the epicardial pacing lead.

It should be understood that the systems, devices, and methods of the invention may be used in setting other than epicardial pacing. Any human, veterinary, or other procedure that benefits from accurate and controlled placement of electrodes in confined spaces may use the systems, devices, and methods of the invention.

### BRIEF DESCRIPTION OF THE DRAWINGS

The foregoing summary and detailed description is better understood when read in conjunction with the accompanying drawings which are included by way of example and not by way of limitation.

FIG. 1 shows a schematic of an exemplary epicardial pacing lead.

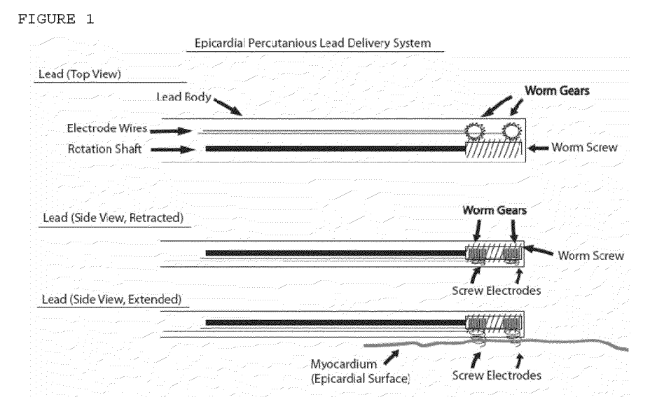

FIG. 2 show a schematic of an exemplary delivery sheath.

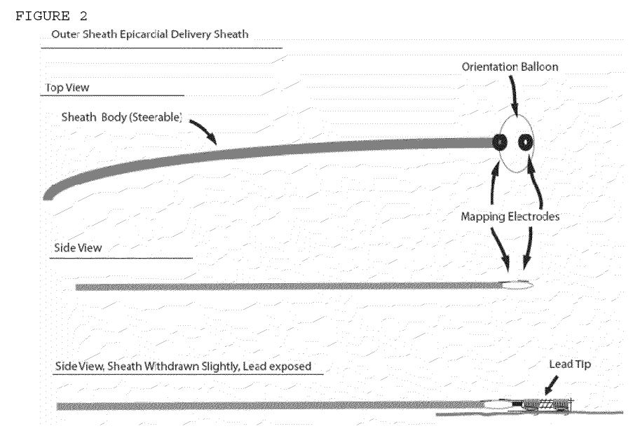

### DETAILED DESCRIPTION OF EMBODIMENTS

The present invention provides systems, devices, and methods for pericardial lead placement without open thorocotomy, through a needle-based percutaneous access into the pericardial space. In some embodiments, the present invention provides a delivery sheath that is steerable and with mapping capabilities, and shaped for optimum navigation within the pericardial space. In some embodiments, the present invention provides a pacing lead that is designed to be placed through the sheath and with screw-out electrodes orthogonal to the catheter shaft for epicardial placement.

In some embodiments, the present invention comprises an epicardial pacing lead configured to be delivered via a delivery sheath. In some embodiments the lead is of a bipolar configuration. In some embodiments, the lead has two or more screw-in electrodes approximately 2-20 mm apart (e.g. approximately 2 mm, 3, mm, 4 mm, 5 mm, 6, mm, 7 mm, 8 mm, 9 mm, 10, mm, 11 mm, 12 mm, 13, mm, 14 mm, 15 mm, 16, mm, 17 mm, 18 mm, 19 mm, or 20 mm apart). In some embodiments, the epicardial pacing lead provides electrodes which are configured to advance in the orthogonal direction to the lead axis via a gearing mechanism (e.g. worm gears), although any suitable deployment mechanism may be used. In some embodiments, positioning of the lead can be optimized by delivery sheath mapping, using mapping electrodes, as well as coronary angiography. In some embodiments, the electrodes are advanced by rotating a drive shaft at the proximal end of the lead. In some embodiments, the rotating drive shaft turns the gearing mechanism (via, for example, a worm screw and worm gears) resulting in both screw-like electrodes to advance into the cardiac muscle on the epicardial surface. In some embodiments the motion of the screw-like electrodes is reversible, so that the electrodes can be withdrawn back into the lead for lead extraction or repositioning. In some embodiments, once the sheath is removed, the proximal end of the lead can be tunneled subcutaneously up to the pacemaker generator, in the standard pectoral position.

In some embodiments, the epicardial pacing lead, described above, is delivered percutaniously to the epicardium, via a delivery sheath with several unique features that allow for epicardial navigation and lead delivery. In some embodiments, the overall shape and general curve is optimized for pericardial manipulation, as will be understood by one of skill in the art. In some embodiments, the sheath is steerable to allow for navigation within the pericardial plane. In some embodiments, the sheath has bidirectional steerablity, with the sheath tip configured to be bendable in the left/right plane via controls at the sheath handle. In some embodiments, the sheath tip has a pair of electrodes with the same spacing as the lead electrodes to allow for “mapping”—to test pacing thresholds and sensing in a fast and non-destructive manner prior to actual delivery of lead. In some embodiments, impedance measurement, made with the mapping electrodes, helps avoid coronary arteries. In some embodiments, an inflatable, flat pancake-shaped orientation balloon at the sheath tip helps maintain correct orientation within the flat pericardial space, so that the lead electrodes deploy in the correct direction, into the epicardium. In some embodiments, the delivery sheath is configured to be removed from the lead. In some embodiments, the sheath is able to be split with a cutter, allowing it to be removed once the lead is in place. In some embodiments, the sheath is slightly larger than the pacing lead, allowing it to pass over the back of the pacing lead without splitting. In some embodiments, the lead comprises a long stylet for control of lead during sheath removal.

In some embodiments, the outer surface of the delivery sheath may be provided with a smooth coating to allow the sheath and pacing lead to more easily move within the epicardium (e.g. TEFLON, HDPE, nylon, PEBAX, PEEK, PTFE, a water-activated lubricant coating, or other suitable materials). In some embodiments, the delivery sheath generally comprises an elongate tubular body with an axial lumen, and is designed to provide percutaneous access to a diagnostic or treatment site in the body (e.g., epicardium). The elongate tubular body has a proximal section (e.g., steerable sheath body) and a distal section (e.g., orientation balloon and mapping electrodes). The relative length of these two sections can be varied according to clinical need, as will be understood by those skilled in the art. In some embodiments, the delivery sheath comprises a thin, smooth and flexible material.

In some embodiments, the present invention finds use in any situation where transvenous delivery of pacing leads is impossible or high risk (e.g. any clinical scenario now where surgical lead placement is considered). In some embodiments, the present invention is used for drug delivery, gene delivery, ablation therapy, endoscopy, etc.

In some embodiments, the pacing lead and/or the delivery sheath provide a mechanism to prevent rotation of the lead within the sheath, the mechanism may include a physical impediment to rotation (e.g. a notch or slit), but is not limited to any particular mechanism.

In some embodiments, the present invention provides a kit comprising one or more pacing leads and one or more delivery sheaths, along with any associated items such as packaging, instructions, ancillary components, etc. A kit may provide one or more pacing leads and one or more delivery sheaths of varying size, gauge, shape, etc. The kit may contain devices of different sizes or shapes that correspond to the range of different patient profiles a treating clinician might encounter. The kit may further comprise written instructions, software, or other materials useful for using or monitoring the systems.

We claim:

1. A system comprising: (a) an epicardial pacing lead, wherein said epicardial pacing lead comprises a bipolar configuration with respect to the main lead axis, two or more electrodes, a mechanism for attaching the electrodes to a tissue surface, and a rotation shaft; and (b) a delivery sheath, wherein said delivery sheath comprises a cylindrical cover, two or more mapping electrodes, and an orientation balloon; wherein said epicardial pacing lead is configured to reside within said delivery sheath, and said delivery sheath is configured to be removed over said epicardial pacing lead.

2. The system of claim 1, wherein said electrodes are configured to extend orthogonally to said main lead axis.

3. The system of claim 1, wherein said electrodes are spaced 5-15 mm apart.

4. The system of claim 1, wherein said electrodes are spaced 10 mm apart.

5. The system of claim 1, wherein said mechanism is configured to advance said electrodes orthogonally to said main lead axis.

6. The system of claim 1, wherein said rotation shaft is configured to actuate said mechanism for attaching the electrodes to a tissue surface.

7. The system of claim 1, wherein said delivery sheath is steerable via controls at the sheath handle.

8. The system of claim 1, wherein said delivery sheath is bidirectionally steerable.

9. The system of claim 1, wherein said mapping electrodes sit above said lead electrodes and have the same spacing as said lead electrodes

10. The system of claim 1, wherein said orientation balloon comprises a flat pancake-shaped balloon at the sheath tip.

11. The system of claim 1, wherein said orientation balloon is configured to maintain the orientation of said sheath within the flat pericardial space.

12. The system of claim 1, wherein said delivery sheath is configured to be removed over said epicardial pacing lead after being split by a sheath cutter.

13. The system of claim 1, wherein said delivery sheath is configured to be larger than epicardial pacing lead, thereby allowing said sheath to be removed backwards over said epicardial pacing lead.

14. A method comprising:
a) inserting a delivery sheath containing an epicardial pacing lead into the pericardium;
b) positioning said delivery sheath in the proper position over the cardiac muscle;
c) testing for said proper position of said delivery sheath using mapping electrodes on the outer surface of said delivery sheath;
d) removing said delivery sheath from epicardial pacing lead; and
e) engaging said epicardial pacing lead with said cardiac muscle by two or more deployable electrodes positioned at the end of said epicardial pacing lead.

15. The method of claim 14, wherein said inserting comprises steering the delivery sheath through the pericardium via handles on the sheath handle.

16. The method of claim 14, further comprising a step between steps (a) and (b) comprising: maintaining proper orientation of said delivery sheath within the flat pericardial space using a flat inflatable orientation balloon at the sheath tip.

17. The method of claim 14, wherein said testing for said proper position of said delivery sheath using mapping electrodes comprises testing pacing thresholds.

18. The method of claim 14, wherein said removing said delivery sheath from epicardial pacing lead comprises splitting said delivery sheath.

19. The method of claim 14, wherein said removing said delivery sheath from epicardial pacing lead comprises withdrawing said delivery backwards over said epicardial pacing lead.

20. The method of claim 14, wherein said engaging said epicardial pacing lead with said cardiac muscle comprises screwing two screw-in electrodes into said cardiac muscle.

21. The method of claim 20, wherein said screw-in electrodes are turned by a gearing mechanism within said epicardial pacing lead.

22. The method of claim 21, wherein said gearing mechanism is turned by a rotation shaft within said epicardial pacing lead.
